(19) United States
(12) Patent Application Publication
(10) Pub. No.: US 2019/0151658 A1
(43) Pub. Date: May 23, 2019

(54) ELECTRICAL PULSE STIMULATION SYSTEM AND CONTROLLING METHOD THEREOF, AND ELECTRICAL PULSE STIMULATION THERAPY

(71) Applicants: Tsinghua University, Beijing (CN); BEIJING PINS MEDICAL CO., LTD, Beijing (CN)

(72) Inventors: FU-MIN JIA, Beijing (CN); JIA-CHU ZHANG, Beijing (CN); SHI-XIU YAO, Beijing (CN); HAO CUI, Beijing (CN); BO-ZHI MA, Beijing (CN); HONG-WEI HAO, Beijing (CN); LU-MING LI, Beijing (CN)

(73) Assignees: Tsinghua University, Beijing (CN); BEIJING PINS MEDICAL CO., LTD, Beijing (CN)

(21) Appl. No.: 16/198,828

(22) Filed: Nov. 22, 2018

(30) Foreign Application Priority Data
Nov. 23, 2017 (CN) 201711181607.4

(51) Int. Cl. A61N 1/36; A61N 1/05; A61N 1/372

(52) U.S. Cl. CPC A61N 1/36007; A61N 1/0551; A61N 1/37235; A61N 1/36135; A61N 1/36125

(57) ABSTRACT

An electrical pulse stimulation system includes a pulse generator, a first lead electrically connected to the pulse generator, and a second lead electrically connected to the pulse generator. The pulse generator is configured to deliver a left sacral nerve electrical pulse stimulation signal to the first lead and deliver a right sacral nerve electrical pulse stimulation signal to the second lead. The pulse generator is further configured to determine and adjust a stimulation quantity proportion of the left sacral nerve electrical pulse stimulation signal and the right sacral nerve electrical pulse stimulation signal according to a sacral nerve data of a patient.

### CROSS-REFERENCE TO RELATED APPLICATIONS

This application claims all benefits accruing under 35 U.S.C. § 119 from China Patent Applications No. 201711181607.4, filed on Nov. 23, 2017 in the China Intellectual Property Office, the content of which is hereby incorporated by reference.

### FIELD

This subject matter herein generally relates to therapy methods and medical devices, especially an electrical pulse stimulation system and controlling method thereof, and an electrical pulse stimulation therapy.

### BACKGROUND

Implantable medical devices, such as cardiac pacemakers, defibrillators, deep brain stimulators, spinal cord stimulators, vagus nerve stimulators, stomach stimulators, muscle stimulators, and peripheral nerve stimulators, have been widely used for disease therapy. The implantable medical device usually includes an internal implantable device and an external controlling device. The internal implantable device and the external controlling device can exchange information through two-way wireless communication. An implantable sacral neuromodulation (SNM) system can be used to treat urinary dysfunction, especially lower urinary tract symptoms, such as overactive bladder syndrome and chronic urinary retention. Implantable sacral neuromodulation devices have been widely applied in clinical practice because of its high efficacy, reversibility, and non-toxic side effects.

However, more and more clinical and anatomical evidences such as clinical neuroanatomy, neurophysiology, and animal experimentation show that the sacral nerve may have variations. The sacral nerve variations include anatomical and functional variations. Because of the anatomical and the functional variations, the sacral nerve may have different responses when the sacral nerve is stimulated by the same electrical pulse stimulation intensity; or in order to obtain the same reaction effect, the sacral nerve may need to be stimulated by a different electrical pulse stimulation intensity. The conventional sacral neuromodulation system only has the function of electrical stimulation of a unilateral sacral nerve, such as S3 nerve root of the left or right side. The conventional sacral neuromodulation system cannot be adapted to the sacral nerve variations or physiological changes once implanted, which has limited the clinical application.

Therefore, there is room for improvement in this art.

### BRIEF DESCRIPTION OF THE DRAWINGS

Implementations are described by way of example only with reference to the attached figures.

FIG. 1 is a schematic view of one embodiment of an electrical pulse stimulation system.

FIG. 2 is a functional diagram of one embodiment of a pulse generator of the electrical pulse stimulation system.

FIG. 3 is a schematic view of one embodiment showing how the electrical pulse stimulation system works.

FIG. 4a is a schematic view of one embodiment of a first kind of an electrical pulse stimulation signal generated by the electrical pulse stimulation system.

FIG. 4b is a schematic view of one embodiment of a second kind of an electrical pulse stimulation signal generated by the electrical pulse stimulation system.

FIG. 4c is a schematic view of one embodiment of a third kind of an electrical pulse stimulation signal generated by the electrical pulse stimulation system.

FIG. 4d is a schematic view of one embodiment of a fourth kind of an electrical pulse stimulation signal generated by the electrical pulse stimulation system.

FIG. 4e is a schematic view of one embodiment of a fifth kind of an electrical pulse stimulation signal generated by the electrical pulse stimulation system.

FIG. 4f is a schematic view of one embodiment of a sixth kind of an electrical pulse stimulation signal generated by the electrical pulse stimulation system.

FIG. 4g is a schematic view of one embodiment of a seventh kind of an electrical pulse stimulation signal generated by the electrical pulse stimulation system.

FIG. 5 is a flow chart of one embodiment of a method for controlling the electrical pulse stimulation signal.

FIG. 6 is a flow chart of another embodiment of a method for controlling the electrical pulse stimulation signal.

FIG. 7 is a flow chart of one embodiment of a method for determining a first left sacral nerve electrical pulse stimulation signal and a first right sacral nerve electrical pulse stimulation signal.

FIG. 8 is a flow chart of one embodiment of a method for determining a first left sacral nerve electrical pulse stimulation signal and a first right sacral nerve electrical pulse stimulation signal.

FIG. 9 is a flow chart of one embodiment of a method for determining a first left sacral nerve electrical pulse stimulation signal and a first right sacral nerve electrical pulse stimulation signal.

FIG. 10 is a flow chart of one embodiment of a method for controlling the electrical pulse stimulation system.

FIG. 11 is a flow chart of another one embodiment of a method for controlling the electrical pulse stimulation system.

FIG. 12 is a flow chart of one embodiment of an electrical pulse stimulation therapy for urinary dysfunction.

### SUMMARY

In one embodiment, an electrical pulse stimulation system is provided. The electrical pulse stimulation system includes: a pulse generator; a first lead electrically connected to the pulse generator; and a second lead electrically connected to the pulse generator; wherein the pulse generator is configured to deliver a left sacral nerve electrical pulse stimulation signal to the first lead and deliver a right sacral nerve electrical pulse stimulation signal to the second lead; and the pulse generator is further configured to determine and adjust a stimulation quantity proportion of the left sacral nerve electrical pulse stimulation signal and the right sacral nerve electrical pulse stimulation signal according to a sacral nerve data of a patient.

The electrical pulse stimulation system further includes an external programmer, wherein the external programmer communicates with the pulse generator and is configured to send an initial sacral nerve data to the pulse generator.

In the electrical pulse stimulation system described above, the pulse generator includes: a processor; a pulse generation circuit electrically connected to the processor; a communication device electrically connected to the processor; a memory electrically connected to the processor; and a coordinator electrically connected to the processor, the first lead, and the second lead; wherein the coordinator is configured to determine a first stimulation quantity proportion of a first left sacral nerve electrical pulse stimulation signal and a first right sacral nerve electrical pulse stimulation signal according to the initial sacral nerve data of the patient, and deliver the first left sacral nerve electrical pulse stimulation signal to the first lead and deliver the first right sacral nerve electrical pulse stimulation signal to the second lead.

In the electrical pulse stimulation system described above, the pulse generator further includes a sensor, the sensor is electrically connected to the processor and configured to detect a real time sacral nerve data of the patient; wherein the coordinator is further configured to determine a second stimulation quantity proportion of a second left sacral nerve electrical pulse stimulation signal and a second right sacral nerve electrical pulse stimulation signal according to the real time sacral nerve data of the patient, and deliver the second left sacral nerve electrical pulse stimulation signal to the first lead and deliver the second right sacral nerve electrical pulse stimulation signal to the second lead.

In the electrical pulse stimulation system described above, the coordinator has a machine learning function so that the coordinator is configured to automatically adjust the second stimulation quantity proportion of the second left sacral nerve electrical pulse stimulation signal and the second right sacral nerve electrical pulse stimulation signal according to the real time sacral nerve data of the patient.

In the electrical pulse stimulation system described above, the second stimulation quantity proportion is different from the first stimulation quantity proportion.

In the electrical pulse stimulation system described above, the coordinator is further configured to apply the first and second left sacral nerve electrical pulse stimulation signals and the first and second right sacral nerve electrical pulse stimulation signals in a preset stimulation sequence.

In the electrical pulse stimulation system described above, the coordinator is further configured to simultaneously apply the first and second left sacral nerve electrical pulse stimulation signals and the first and second right sacral nerve electrical pulse stimulation signals.

In the electrical pulse stimulation system described above, the coordinator is further configured to alternately apply the first and second left sacral nerve electrical pulse stimulation signals and the first and second right sacral nerve electrical pulse stimulation signals.

In the electrical pulse stimulation system described above, the coordinator is further configured to randomly apply the first and second left sacral nerve electrical pulse stimulation signals and the first and second right sacral nerve electrical pulse stimulation signals.

In one embodiment, a method for controlling a pulse generator of an electrical pulse stimulation system is provided. The method includes: obtaining an initial sacral nerve data; determining a first left sacral nerve electrical pulse stimulation signal and a first right sacral nerve electrical pulse stimulation signal according to the initial sacral nerve data; and applying the first left sacral nerve electrical pulse stimulation signal and the first right sacral nerve electrical pulse stimulation signal.

The method further includes obtaining a real time sacral nerve data during applying the first left sacral nerve electrical pulse stimulation signal and the first right sacral nerve electrical pulse stimulation signal, and the real time sacral nerve data is different from the initial sacral nerve data; determining a second left sacral nerve electrical pulse stimulation signal and a second right sacral nerve electrical pulse stimulation signal according to the real time sacral nerve data; and applying the second left sacral nerve electrical pulse stimulation signal and the second right sacral nerve electrical pulse stimulation signal.

In the method described above, the determining the first left sacral nerve electrical pulse stimulation signal and the first right sacral nerve electrical pulse stimulation signal according to the initial sacral nerve data includes: determining the total stimulation quantity of the sacral nerve electrical pulse stimulation according to the initial sacral nerve data; setting a first stimulation quantity of the first left sacral nerve electrical pulse stimulation signal, wherein the first stimulation quantity is less than the total stimulation quantity; and setting a second stimulation quantity of the first right sacral nerve electrical pulse stimulation signal according to the first stimulation quantity and the total stimulation quantity.

In the method described above, the determining the first left sacral nerve electrical pulse stimulation signal and the first right sacral nerve electrical pulse stimulation signal according to the initial sacral nerve data includes: determining the total stimulation quantity of the sacral nerve electrical pulse stimulation according to the initial sacral nerve data; setting a first stimulation quantity proportion of the first left sacral nerve electrical pulse stimulation signal and the first right sacral nerve electrical pulse stimulation signal; and setting a first stimulation quantity of the first left sacral nerve electrical pulse stimulation signal and a second stimulation quantity of the first right sacral nerve electrical pulse stimulation signal according to the first stimulation quantity proportion and the total stimulation quantity.

In the method described above, the determining the first left sacral nerve electrical pulse stimulation signal and the first right sacral nerve electrical pulse stimulation signal according to the initial sacral nerve data includes: S201, applying a first detection electrical pulse stimulation signal to one of a left sacral nerve and a right sacral nerve according to the initial sacral nerve data; S202, determining whether the first detection electrical pulse stimulation signal achieves a sensory threshold of the corresponding sacral nerve being stimulated by the first detection electrical pulse stimulation signal, when the first detection electrical pulse stimulation signal achieves the sensory threshold of the corresponding sacral nerve, stopping applying and saving the first detection electrical pulse stimulation signal, and go to S203; when the first detection electrical pulse stimulation signal does not achieve the sensory threshold of the corresponding sacral nerve, adjusting the first detection electrical pulse stimulation signal, and go back to S201; S203, applying a second detection electrical pulse stimulation signal to the other one of the left sacral nerve and the right sacral nerve according to the first sacral nerve data; S204, determining whether the second detection electrical pulse stimulation signal achieves the sensory threshold of the corresponding sacral nerve being stimulated by the second detection electrical pulse stimulation signal, when the second detection electrical pulse stimulation signal achieves the sensory threshold of the corresponding sacral nerve, stopping applying and saving the second detection electrical pulse stimulation signal, and go to S205; when the second detection electrical pulse stimulation signal does not achieve the sensory threshold of the corresponding sacral nerve, adjusting the second detection electrical pulse stimulation signal, and go back to S203; and S205, determining the first left sacral nerve electrical pulse stimulation signal and the first right sacral nerve electrical pulse stimulation signal according to the first detection electrical pulse stimulation signal and the second detection electrical pulse stimulation signal.

In one embodiment, an electrical pulse stimulation therapy for urinary dysfunction by an electrical pulse stimulation system is provided. The electrical pulse stimulation therapy includes: obtaining an initial sacral nerve data before applying any electrical pulse stimulation to the patient; determining a first left sacral nerve electrical pulse stimulation signal and a first right sacral nerve electrical pulse stimulation signal according to the initial sacral nerve data; and applying the first left sacral nerve electrical pulse stimulation signal and the first right sacral nerve electrical pulse stimulation signal to sacral nerve of the patient.

The electrical pulse stimulation therapy further includes obtaining a real time sacral nerve data during applying the first left sacral nerve electrical pulse stimulation signal and the first right sacral nerve electrical pulse stimulation signal to the sacral nerve of the patient, and the real time sacral nerve data is different from the initial sacral nerve data; determining a second left sacral nerve electrical pulse stimulation signal and a second right sacral nerve electrical pulse stimulation signal according to the real time sacral nerve data; and applying the second left sacral nerve electrical pulse stimulation signal and the second right sacral nerve electrical pulse stimulation signal to the sacral nerve of the patient.

In the electrical pulse stimulation therapy described above, the determining the first left sacral nerve electrical pulse stimulation signal and the first right sacral nerve electrical pulse stimulation signal according to the initial sacral nerve data includes: determining the total stimulation quantity of the sacral nerve electrical pulse stimulation according to the initial sacral nerve data; setting a first stimulation quantity of the first left sacral nerve electrical pulse stimulation signal, wherein the first stimulation quantity is less than the total stimulation quantity; and setting a second stimulation quantity of the first right sacral nerve electrical pulse stimulation signal according to the first stimulation quantity and the total stimulation quantity.

In the electrical pulse stimulation therapy described above, the determining the first left sacral nerve electrical pulse stimulation signal and the first right sacral nerve electrical pulse stimulation signal according to the initial sacral nerve data includes: determining the total stimulation quantity of the sacral nerve electrical pulse stimulation according to the initial sacral nerve data; setting a first stimulation quantity proportion of the first left sacral nerve electrical pulse stimulation signal and the first right sacral nerve electrical pulse stimulation signal; and setting a first stimulation quantity of the first left sacral nerve electrical pulse stimulation signal and a second stimulation quantity of the first right sacral nerve electrical pulse stimulation signal according to the first stimulation quantity proportion and the total stimulation quantity.

In the electrical pulse stimulation therapy described above, the determining the first left sacral nerve electrical pulse stimulation signal and the first right sacral nerve electrical pulse stimulation signal according to the initial sacral nerve data includes: S201, applying a first detection electrical pulse stimulation signal to one of a left sacral nerve and a right sacral nerve according to the initial sacral nerve data; S202, determining whether the first detection electrical pulse stimulation signal achieves a sensory threshold of the corresponding sacral nerve being stimulated by the first detection electrical pulse stimulation signal, when the first detection electrical pulse stimulation signal achieves the sensory threshold of the corresponding sacral nerve, stopping applying and saving the first detection electrical pulse stimulation signal, and go to S203; when the first detection electrical pulse stimulation signal does not achieve the sensory threshold of the corresponding sacral nerve, adjusting the first detection electrical pulse stimulation signal, and go back to S201; S203, applying a second detection electrical pulse stimulation signal to the other one of the left sacral nerve and the right sacral nerve according to the first sacral nerve data; S204, determining whether the second detection electrical pulse stimulation signal achieves the sensory threshold of the corresponding sacral nerve being stimulated by the second detection electrical pulse stimulation signal, when the second detection electrical pulse stimulation signal achieves the sensory threshold of the corresponding sacral nerve, stopping applying and saving the second detection electrical pulse stimulation signal, and go to S205; when the second detection electrical pulse stimulation signal does not achieve the sensory threshold of the corresponding sacral nerve, adjusting the second detection electrical pulse stimulation signal, and go back to S203; and S205, determining the first left sacral nerve electrical pulse stimulation signal and the first right sacral nerve electrical pulse stimulation signal according to the first detection electrical pulse stimulation signal and the second detection electrical pulse stimulation signal.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Sacral neuromodulation is a technique for treating micturition dysfunctions. Sacral neuromodulation usually artificially activates the excitatory neural pathways or inhibitory neural pathways to modulate the function of the bladder, external urethral sphincter and pelvic floor muscles by applying the intermittent persistent short-frequency pulse stimulation current to the sacral nerve. Currently, the conventional sacral neuromodulation therapy cannot benefit all patients.

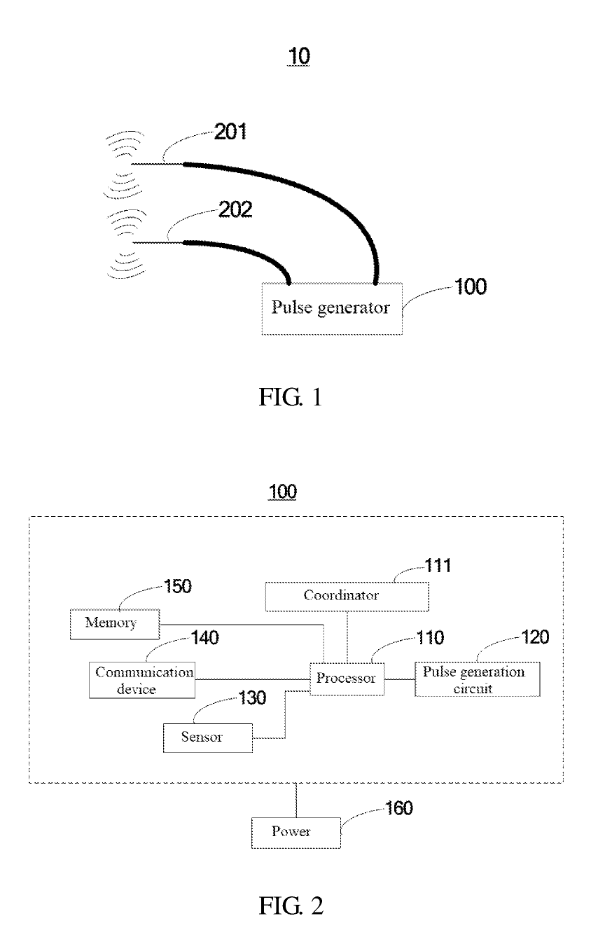

As shown in FIG. 1, in order to synthetically adjust the electrical pulse stimulation signal according to the patients' sacral nerve anatomical and functional variations and benefit more patients from the sacral neuromodulation, an electrical pulse stimulation system 10 is provided for sacral neuromodulation. In one embodiment, the electrical pulse stimulation system 10 includes a pulse generator 100, a first lead 201, and a second lead 202. The first lead 201 and the second lead 202 can be different kinds or types of leads.

The pulse generator 100 is respectively electrically connected to the first lead 201 and the second lead 202. The pulse generator 100 can be electrically connected to the first lead 201 and the second lead 202 by wires. The pulse generator 100 at least has the variable frequency stimulation mode to generate the variable frequency electrical pulse stimulation signal. The variable frequency electrical pulse stimulation signal includes at least two or three alternate electrical stimulation pulse trains at two or three different frequencies to form a plurality of pulse train periods. The pulse generator 100 can deliver the electrical pulse stimulation signals to the first lead 201 and the second lead 202 at different proportion.

The electrical pulse stimulation signals at different proportion means the electrical pulse stimulation signals have different stimulation quantity. The stimulation quantity depends on voltage, current, pulse width, and frequency. The first lead 201 can includes a plurality of first electrodes that is to be stimulated, and the stimulation quantity of the first lead 201 can be the sum of the stimulation quantity of the plurality of first electrodes that is to be stimulated. The second lead 202 can also include a plurality of second electrodes that is to be stimulated, and the stimulation quantity of the second lead 202 can be the sum of the stimulation quantity of the plurality of second electrodes that is to be stimulated. The first lead 201 or the second lead 202 has more electrodes, the larger area of the nervous system can be stimulated. It is not limited that one of the first lead 201 and the second lead 202 has to be used to stimulate left sacral nerve and the other one has to be used to stimulate right sacral nerve. The first lead 201 can be used to stimulate left sacral nerve or the right sacral nerve. The second lead 202 can also be used to stimulate left sacral nerve or the right sacral nerve. The first lead 201 and the second lead 202 can also be simultaneously used to stimulate the different nervous ramifications of the same one of the left sacral nerve and the right sacral nerve. To different nervous ramifications of the same sacral nerve, the first lead 201 and the second lead 202 can apply different or the same electrical pulse stimulation signals.

In one embodiment, both the first lead 201 and the second lead 202 can simultaneously apply the electrical pulse stimulation signals to the left sacral nerve. In one embodiment, both the first lead 201 and the second lead 202 can simultaneously apply the electrical pulse stimulation signals to the right sacral nerve. In one embodiment, one of the first lead 201 and the second lead 202 applies an electrical pulse stimulation signal to the left sacral nerve, and the other one of the first lead 201 and the second lead 202 applies an electrical pulse stimulation signal to the right sacral nerve. For example, the first lead 201 applies a first electrical pulse stimulation signal to the left S2 sacral nerve, and the second lead 202 applies a second electrical pulse stimulation signal to the right S4 sacral nerve. For example, the first lead 201 applies a third electrical pulse stimulation signal to the left S3 sacral nerve, and the second lead 202 applies a fourth electrical pulse stimulation signal to the left S4 sacral nerve.

The first lead 201 and the second lead 202 can be understood as dual channel leads for applying different electrical pulse stimulation signals, such as a first left sacral nerve electrical pulse stimulation signal and first right sacral nerve electrical pulse stimulation signal. The dual channel leads can deliver the electrical pulse stimulation signals generated by the pulse generator 100 and stimulate the sacral nerve of the patient. The stimulation quantity proportion of the electrical pulse stimulation signals inputted to dual channel leads from the pulse generator 100 is adjustable. In one embodiment, the first left sacral nerve electrical pulse stimulation signal can be applied to the left sacral nerve or the right sacral nerve. Also, the first right sacral nerve electrical pulse stimulation signal can be applied to the right sacral nerve or the left sacral nerve.

The pulse generator 100 can deliver the first left sacral nerve electrical pulse stimulation signal to the first lead 201. The pulse generator 100 can also deliver the first right sacral nerve electrical pulse stimulation signal to the second lead 202. The pulse generator 100 can determine and adjust the stimulation quantity proportion of the first left sacral nerve electrical pulse stimulation signal and the first right sacral nerve electrical pulse stimulation signal according to the sacral nerve data of patient.

The first left sacral nerve electrical pulse stimulation signal and the first right sacral nerve electrical pulse stimulation signal can be determined and adjusted according to the real time sacral nerve data of patient such as response threshold. The response threshold can include the left sacral nerve sensory threshold, the right sacral nerve sensory threshold, the left sacral nerve motor threshold, and the right sacral nerve motor threshold. The response threshold can also be other physiological characteristic of the patients. The first left sacral nerve electrical pulse stimulation signal and the first right sacral nerve electrical pulse stimulation signal can be interrelated with each other. The pulse generator 100 determines and adjusts the stimulation quantity proportion of the first left sacral nerve electrical pulse stimulation signal applied to the left sacral nerve and the first right sacral nerve electrical pulse stimulation signal applied to the right sacral nerve according to the real time sacral nerve data of patient. The sacral nerve data can include the left sacral nerve sensory threshold, the right sacral nerve sensory threshold, the left sacral nerve motor threshold, and the right sacral nerve motor threshold. The sacral nerve data detected before performing the electrical pulse stimulation is defined as the first sacral nerve data. The first sacral nerve data can include the left sacral nerve sensory threshold, the right sacral nerve sensory threshold, the left sacral nerve motor threshold, and the right sacral nerve motor threshold. The sacral nerve data can also be another physiological characteristic of the patient. In one embodiment, the sensory threshold is represented by Tsen, and the motor threshold is represented by Tmor.

The sensory threshold is the weakest electrical pulse stimulation that the patient can feel the contraction of the penis root or vagina, rectal traction, following with or without extension to the scrotum or labium anteriorly, which are caused by the current stimulation. The motor threshold is the weakest electrical pulse stimulation that the patient has wind box movement in the perineal region of the pelvic floor, hallux plantar flexor reflex of lower limbs, following with or without another plantar flexor reflex. Under different morbid states or different electrical pulse stimulations, the left sacral nerve sensory threshold and the right sacral nerve sensory threshold are usually different, and the left sacral nerve motor threshold and the right sacral nerve motor threshold are also usually different. Thus, the left sacral nerve sensory threshold, the right sacral nerve sensory threshold, the left sacral nerve motor threshold, and the right sacral nerve motor threshold of different patients should be obtained. Before applying the electrical pulse stimulation, the sensory threshold and the motor threshold can be the weakest electrical pulse stimulation that is acceptable to the patient within a small range. The sensory threshold and the motor threshold are real time changed and would gradually increase as the extension of the electrical pulse stimulation time. The different nervous ramifications of the same sacral nerve may have different sensory thresholds and different motor thresholds. Different patients may have different sensory thresholds and different motor thresholds.

In one embodiment, the first lead 201 and the second lead 202 can apply the electrical pulse stimulation signals in different stimulation quantity proportion to the sacral nerve. After applying the different electrical pulse stimulation signals to the sacral nerve, the patient may have the different stimulation responses. The different stimulation reaction includes the change of sensory threshold and motor threshold. The real time change of the sensory threshold and motor threshold of the patient can be obtained, and then the electrical pulse stimulation signal applied to the left sacral nerve and the electrical pulse stimulation signal applied to the right sacral nerve can be adjusted in real time according to the real time change of the sensory threshold and motor threshold. The left sacral nerve motor threshold, the left sacral nerve sensory threshold, the right sacral nerve motor threshold, and the right sensory threshold can have an initial value before applying electrical pulse stimulation and form an initial sacral nerve data. The initial sacral nerve data detected before applying electrical pulse stimulation is defined as the first sacral nerve data. The real time sacral nerve data detected during applying electrical pulse stimulation is defined as the second sacral nerve data. The left sacral nerve motor threshold, the left sacral nerve sensory threshold, the right sacral nerve motor threshold, and the right sensory threshold would change after applying the different electrical pulse stimulation to the sacral nerve. Namely, the patient's nerve anatomy or function distribution has a variation. The electrical pulse stimulation signals applied to the sacral nerve via the first lead 201 and the second lead 202 can synthetically adjusted so that the left sacral nerve and the right sacral nerve can be stimulated by different the electrical pulse stimulation signals. The electrical pulse stimulation signals applied to the sacral nerve via the first lead 201 and the second lead 202 can be synthetically adjusted so that the different nervous ramification of the left sacral nerve or the right sacral nerve can be stimulated by different the electrical pulse stimulation signals. Thus, the electrical pulse stimulation system 10 can synthetically adjust the electrical pulse stimulation signal according to the patients' nerve anatomy or function distribution variation.

As shown in FIG. 2, in one embodiment, the pulse generator 100 includes a processor 110, a coordinator 111, a pulse generation circuit 120, a sensor 130, a communication device 140, and a memory 150.

The pulse generation circuit 120, the sensor 130, the communication device 140, and the memory 150 are electrically connected to the processor 110 by wire or wireless. The pulse generation circuit 120 is configured to generate the electrical pulse stimulation signal. The sensor 130 is configured to detect the sacral nerve data of the patient, such as the left sacral nerve sensory threshold, the right sacral nerve sensory threshold, the left sacral nerve motor threshold, and the right sacral nerve motor threshold.

The coordinator 111 is electrically connected to the processor 110 by wire or wirelessly. The coordinator 111 is configured to determine and adjust the stimulation quantity proportion of the first left sacral nerve electrical pulse stimulation signal and the first right sacral nerve electrical pulse stimulation signal according to the first sacral nerve data of the patient. The coordinator 111 is electrically connected to the first lead 201 and the second lead 202. The coordinator 111 delivers the first left sacral nerve electrical pulse stimulation signal to the first lead 201, and delivers the first right sacral nerve electrical pulse stimulation signal to the second lead 202.

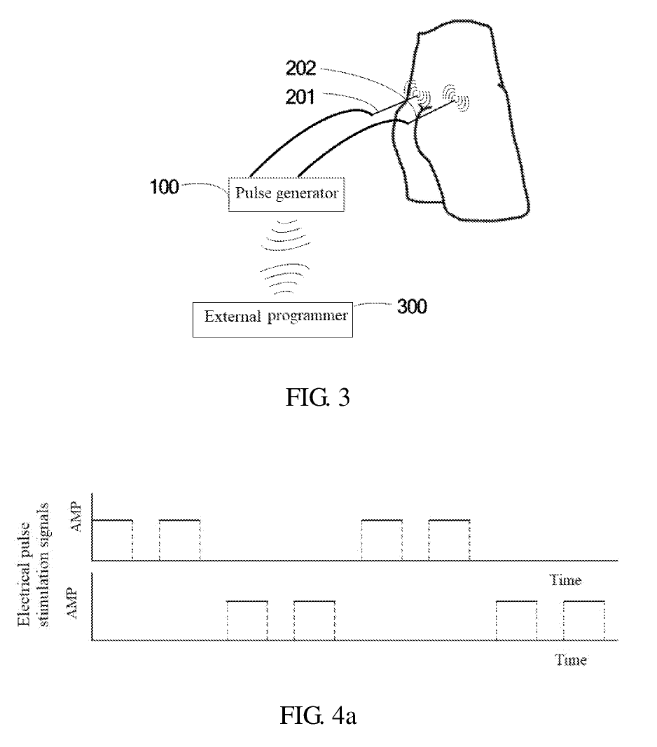

The coordinator 111 can apply the electrical pulse stimulation signals in different stimulation quantity proportion to the left sacral nerve and the right sacral nerve according to the first sacral nerve data, such as the initial left sacral nerve sensory threshold, the initial right sacral nerve sensory threshold, the initial left sacral nerve motor threshold, and the initial right sacral nerve motor threshold. The coordinator 111 can further adjust the electrical pulse stimulation signals applied to the left sacral nerve and the right sacral nerve according to the real time sacral nerve data detected by the sensor 130, such as the real time left sacral nerve sensory threshold, the real time right sacral nerve sensory threshold, the real time left sacral nerve motor threshold, or the real time right sacral nerve motor threshold. The work parameter of the coordinator 111 can be set through the external programmer 300 as shown in FIG. 3 by the doctor. Thus, the coordinator 111 can be manually controlled to apply different the electrical pulse stimulation signals to the sacral nerve of the patient via the first lead 201 and the second lead 202. The coordinator 111 can also have the machine learning function so that the coordinator 111 can automatically apply different electrical pulse stimulation signals to the sacral nerve of the patient via the first lead 201 and the second lead 202. Thus, the electrical pulse stimulation system 10 can have more sensitive feedback to the sensory threshold and the motor threshold. The coordinator 111 can adjust the stimulation quantity proportion of the first left sacral nerve electrical pulse stimulation signal and the first right sacral nerve electrical pulse stimulation signal according to the patient's real time sensory threshold and the real time motor threshold. Thus, the pulse generator 100 can automatically apply different the electrical pulse stimulation signals to the sacral nerve of the patient according to the stored relationship between the threshold (sensory threshold and the motor threshold) and the stimulation quantity proportion.

The coordinator 111 can apply the first left sacral nerve electrical pulse stimulation signal and the first right sacral nerve electrical pulse stimulation signal in a preset stimulation sequence.

In one embodiment, the preset stimulation sequence can include simultaneously applying the first left sacral nerve electrical pulse stimulation signal and the first right sacral nerve electrical pulse stimulation signal, alternately applying the first left sacral nerve electrical pulse stimulation signal and the first right sacral nerve electrical pulse stimulation signal, or randomly applying the first left sacral nerve electrical pulse stimulation signal and the first right sacral nerve electrical pulse stimulation signal. For example, the coordinator 111 can first determine the stimulation quantity proportion of the first left sacral nerve electrical pulse stimulation signal and the first right sacral nerve electrical pulse stimulation signal, and then simultaneously apply the first left sacral nerve electrical pulse stimulation signal and the first right sacral nerve electrical pulse stimulation signal in the predetermined stimulation quantity proportion to the left sacral nerve. For example, the coordinator 111 can first determine the stimulation quantity proportion of the first left sacral nerve electrical pulse stimulation signal and the first right sacral nerve electrical pulse stimulation signal, and then simultaneously apply the first left sacral nerve electrical pulse stimulation signal and the first right sacral nerve electrical pulse stimulation signal in the predetermined stimulation quantity proportion to the right sacral nerve. For example, the coordinator 111 can first determine the stimulation quantity proportion of the first left sacral nerve electrical pulse stimulation signal and the first right sacral nerve electrical pulse stimulation signal, and then simultaneously apply the first left sacral nerve electrical pulse stimulation signal in the predetermined to the left sacral nerve and apply the first right sacral nerve electrical pulse stimulation signal in the predetermined to the right sacral nerve.

For example, the coordinator 111 can first determine the stimulation quantity proportion of the first left sacral nerve electrical pulse stimulation signal and the first right sacral nerve electrical pulse stimulation signal, and then alternately apply the first left sacral nerve electrical pulse stimulation signal and the first right sacral nerve electrical pulse stimulation signal in the predetermined stimulation quantity proportion to the left sacral nerve. For example, the coordinator 111 can first determine the stimulation quantity proportion of the first left sacral nerve electrical pulse stimulation signal and the first right sacral nerve electrical pulse stimulation signal, and then alternately apply the first left sacral nerve electrical pulse stimulation signal and the first right sacral nerve electrical pulse stimulation signal in the predetermined stimulation quantity proportion to the right sacral nerve. For example, the coordinator 111 can first determine the stimulation quantity proportion of the first left sacral nerve electrical pulse stimulation signal and the first right sacral nerve electrical pulse stimulation signal, and then simultaneously alternately apply the first left sacral nerve electrical pulse stimulation signal and the first right sacral nerve electrical pulse stimulation signal in the predetermined stimulation quantity proportion to the left sacral nerve and apply the first left sacral nerve electrical pulse stimulation signal and the first right sacral nerve electrical pulse stimulation signal in the predetermined stimulation quantity proportion to the right sacral nerve.

In one embodiment, the first left sacral nerve electrical pulse stimulation signal and the first right sacral nerve electrical pulse stimulation signal can be an order electrical pulse stimulation signal in a short term, but a disorder electrical pulse stimulation signal in a long term. For example, the stimulation quantity proportion of the first left sacral nerve electrical pulse stimulation signal and the first right sacral nerve electrical pulse stimulation signal can be 1:1, or 6:4. When the stimulation quantity proportion of the first left sacral nerve electrical pulse stimulation signal and the first right sacral nerve electrical pulse stimulation signal is 6:4, the rate of the stimulation quantity applied to the left sacral nerve at the first time, the stimulation quantity applied to the right sacral nerve at the first time, the stimulation quantity applied to the left sacral nerve at the second time, the stimulation quantity applied to the right sacral nerve at the second time can be 3:2:3:2, 2:2:4:2, or 1:2:5:2. The order electrical pulse stimulation signal refers that it is predictable that which one of the first lead 201 and the second lead 202 would be used to apply the electrical pulse stimulation signal next time. The disorder electrical pulse stimulation signal refers that it is not predictable that which one of the first lead 201 and the second lead 202 would be used to apply the electrical pulse stimulation signal next time.

As discussed above, even if the stimulation quantity proportion of the first left sacral nerve electrical pulse stimulation signal and the first right sacral nerve electrical pulse stimulation signal is constant, the stimulation sequence may be different. For example, when the stimulation quantity proportion of the first left sacral nerve electrical pulse stimulation signal and the first right sacral nerve electrical pulse stimulation signal is 6:4, the first left sacral nerve electrical pulse stimulation signal and the first right sacral nerve electrical pulse stimulation signal can be applied simultaneously or applied alternately. The stimulation sequence itself may have clinical significance. The constant stimulation sequence may result in adaptability to stimulation therapy.

In one embodiment, the first left sacral nerve electrical pulse stimulation signal and the first right sacral nerve electrical pulse stimulation signal are alternately applied. In one embodiment, the first left sacral nerve electrical pulse stimulation signal and the first right sacral nerve electrical pulse stimulation signal are simultaneously applied. The stimulation sequences of alternately applying electrical pulse stimulation signals and simultaneously applying electrical pulse stimulation signals may cause different reactions. The alternately applying electrical pulse stimulation signals can stimulate the nerve system more completely. The simultaneously applying electrical pulse stimulation signals can make the body react to the electrical pulse stimulation signals more rapidly, and faster reaction speed and faster response speed can result in an obvious improvement of the therapy effect. If the electrical pulse stimulation signals cause a bad result to the patient, the body may react with different sensory threshold and the motor threshold.

In one embodiment, the electrical pulse stimulation system 10 further comprises an external programmer 300 as shown in FIG. 3. The communication device 140 is electrically connected to the external programmer 300 by wireless. The external programmer 300 communicates with the processor 110 through the communication device 140. The pulse generator 100 is electrically connected to the external programmer 300 by wireless. The external programmer 300 can adjust the work parameter of the pulse generator 100, such as voltage/current, pulse width, frequency, so that the pulse generator 100 apply the first left sacral nerve electrical pulse stimulation signal and the first right sacral nerve electrical pulse stimulation signal in different stimulation quantity proportion to the sacral nerve via the first lead 201 and the second lead 202. The external programmer 300 sends the command signal to the pulse generator 100, the pulse generator 100 adjusts the work parameter according to the command signal and applies the first left sacral nerve electrical pulse stimulation signal and the first right sacral nerve electrical pulse stimulation signal in different stimulation quantity proportion to the sacral nerve via the first lead 201 and the second lead 202. The external programmer 300 can send the command signal to and receive the data from the pulse generator 100. The external programmer 300 can display the work parameter input by the doctor and also display the work parameter receive from the pulse generator 100.

In one embodiment, the memory 150 can store the physiological characteristic of the patient in initial state before applying the electrical pulse stimulation signal to the patient. The physiological characteristic includes the initial left sacral nerve initial motor threshold, the initial left sacral nerve initial sensory threshold, the initial right sacral nerve initial motor threshold, and the initial right initial sensory threshold. The memory 150 can store real time physiological characteristic of the patient detected by the sensor 130 during applying the electrical pulse stimulation signal to the patient, such as the real time left sacral nerve motor threshold, the real time left sacral nerve sensory threshold, the real time right sacral nerve motor threshold, and the real time right sensory threshold. The memory 150 can also store the data including stimulation program group. The stimulation program group is configured to control the first left sacral nerve electrical pulse stimulation signal and the first right sacral nerve electrical pulse stimulation signal that are deliver from the pulse generator 100 to the first lead 201 and the second lead 202. The stimulation program group includes the work parameters of the pulse generator 100, such as voltage, current, pulse width, frequency, and cycle mode and so on. The stimulation program group can also include the relationship of the work parameters and the threshold (sensory threshold and the motor threshold). The work parameter of the pulse generator 100 can be selected or adjusted according to different stimulation quantity or different stimulation quantity proportion. For example, the stimulation program group includes applying the electrical pulse stimulation signal with a frequency of 5 kHz, a pulse width 50 microseconds, and a current 6 milliampere (mA), to the left sacral nerve via the first lead 201. The stimulation program group can be determined according to the type and deliver efficiency of the pulse generator 100.

In one embodiment, the communication device 140 is electrically connected to the external programmer 300 wirelessly. The communication device 140 is configured for the communication between the processor 110 and the external programmer 300.

The communication device 140 can be electrically connected to the external programmer 300 by the third generation (3G) mobile communication technology or the fourth generation (4G) mobile communication technology. The third generation mobile communication technology is the cellular mobile communication technology supporting high-speed data transmission. The third generation mobile communication technology can simultaneously deliver the voice and data information with a data transmission speed above hundreds of kbps. The fourth generation mobile communication technology includes TD-LTE and FDD-LTE. The data transmission speed of the fourth generation mobile communication technology above the 100 Mbit/s, which is 10 thousand times of the mobile phone data transmission speed and 50 times of the 3G mobile phone. In one embodiment, the 3G/4G mobile communication technology is used to achieve the high speed and high efficient wireless communication.

The sensor 130 of the pulse generator 100 is configured to detect the left sacral nerve motor threshold, the left sacral nerve sensory threshold, the right sacral nerve motor threshold, and the right sensory threshold in real time. When the motor threshold and the sensory threshold are changed, the coordinator 111 of the pulse generator 100 delivers a second left sacral nerve electrical pulse stimulation signal to the first lead 201, and delivers a second right sacral nerve electrical pulse stimulation signal to the second lead 202. The stimulation quantity proportion of the second left sacral nerve electrical pulse stimulation signal and the second right sacral nerve electrical pulse stimulation signal is different from the stimulation quantity proportion of the first left sacral nerve electrical pulse stimulation signal and the first right sacral nerve electrical pulse stimulation signal.

In use, more electrical pulse stimulation signals can be applied by the coordinator 111 of the pulse generator 100 according to the changes of the motor threshold and the sensory threshold. For example, the third left sacral nerve electrical pulse stimulation signal can be applied to the left sacral nerve via the first lead 201, and the third right sacral nerve electrical pulse stimulation signal can be applied to the right sacral nerve via the second lead 202. The stimulation quantity proportion of the third left sacral nerve electrical pulse stimulation signal and the third right sacral nerve electrical pulse stimulation signal is different from the stimulation quantity proportion of the second left sacral nerve electrical pulse stimulation signal and the second right sacral nerve electrical pulse stimulation signal. Furthermore, the stimulation sequence of the third left sacral nerve electrical pulse stimulation signal and the third right sacral nerve electrical pulse stimulation signal is different from the stimulation sequence of the second left sacral nerve electrical pulse stimulation signal and the second right sacral nerve electrical pulse stimulation signal. For example, both the second left sacral nerve electrical pulse stimulation signal and the second right sacral nerve electrical pulse stimulation signal are applied to the right sacral nerve. Thus, the stimulation sequence of the pulse generator 100 can also be adjusted according to the changes of the motor threshold and the sensory threshold.

In one embodiment, the sensor 130 can detect the second sacral nerve data of the patient. The coordinator 111 can deliver the second left sacral nerve electrical pulse stimulation signal to the first lead 201 according to the second sacral nerve data, and delivers the second right sacral nerve electrical pulse stimulation signal to the second lead 202 according to the second sacral nerve data. The coordinator 111 can further adjust the stimulation quantity proportion of the second left sacral nerve electrical pulse stimulation signal and the second right sacral nerve electrical pulse stimulation signal according to the second sacral nerve data.

The second sacral nerve data can be the motor threshold or the sensory threshold detected by the sensor 130 during the operation or use of the electrical pulse stimulation system 10. The time point and frequency for obtaining the real time nerve data is not limited and can be selected according to need of the patient or the medical means. The second sacral nerve data can be different from the first sacral nerve data. For example, in the initial state of before implanting the electrical pulse stimulation system 10, the first sacral nerve data are detected first. Then, after implanting the electrical pulse stimulation system 10, the first left sacral nerve electrical pulse stimulation signal is applied to the left sacral nerve via the first lead 201 according to the first sacral nerve data, and the first right sacral nerve electrical pulse stimulation signal is applied to the right sacral nerve via the second lead 202 according to the first sacral nerve data. After applying the first left sacral nerve electrical pulse stimulation signal and the first right sacral nerve electrical pulse stimulation signal for a period of time, the body of the patient would have a reaction. Then, the second sacral nerve data are detected during applying the first left sacral nerve electrical pulse stimulation signal and the first right sacral nerve electrical pulse stimulation signal. When the second sacral nerve data is different from the first sacral nerve data, the first left sacral nerve electrical pulse stimulation signal is adjusted to the second left sacral nerve electrical pulse stimulation signal, and the first right sacral nerve electrical pulse stimulation signal is adjusted to the second right sacral nerve electrical pulse stimulation signal. The second left sacral nerve electrical pulse stimulation signal is applied to the left sacral nerve via the first lead 201 according to the second sacral nerve data, and the second right sacral nerve electrical pulse stimulation signal is applied to the right sacral nerve via the second lead 202 according to the second sacral nerve data. Thus, the therapy to the sacral nerve has been adjusted according to the second sacral nerve data.

As shown in FIG. 3, the electrical pulse stimulation system 10 is used to stimulate the left sacral nerve and the right sacral nerve. Before implanting the electrical pulse stimulation system 10, the physical examination is performed on the patient to obtain the physiological characteristic, and the physiological characteristic is stored in the memory 150 of the pulse generator 100. The physiological characteristic is the first sacral nerve data and includes the initial left sacral nerve initial motor threshold, the initial left sacral nerve initial sensory threshold, the initial right sacral nerve initial motor threshold, and the initial right initial sensory threshold. The coordinator 111 determines the stimulation quantity proportion of the first left sacral nerve electrical pulse stimulation signal and the first right sacral nerve electrical pulse stimulation signal according to the initial motor threshold and the initial sensory threshold, applies the first left sacral nerve electrical pulse stimulation signal to the left sacral nerve via the first lead 201, and applies the first right sacral nerve electrical pulse stimulation signal to the right sacral nerve via the second lead 202.

Then, the sensor 130 detects the second motor threshold and the second sensory threshold during the patient is stimulated by the first left sacral nerve electrical pulse stimulation signal and the first right sacral nerve electrical pulse stimulation signal of the electrical pulse stimulation system 10. The coordinator 111 determine the stimulation quantity proportion of the second left sacral nerve electrical pulse stimulation signal and the second right sacral nerve electrical pulse stimulation signal according to the second motor threshold and the second sensory threshold the stimulation state, applies the second left sacral nerve electrical pulse stimulation signal to the left sacral nerve via the first lead 201, and applies the second right sacral nerve electrical pulse stimulation signal to the right sacral nerve via the second lead 202. The stimulation quantity proportion of the second left sacral nerve electrical pulse stimulation signal and the second right sacral nerve electrical pulse stimulation signal can be same as or different from the stimulation quantity proportion of the first left sacral nerve electrical pulse stimulation signal and the first right sacral nerve electrical pulse stimulation signal.

The second left sacral nerve electrical pulse stimulation signal and the second right sacral nerve electrical pulse stimulation signal are determined according to the second motor threshold and the second sensory threshold in real time. The change of the first sacral nerve data and the second sacral nerve data is automatically or manually obtained by detecting the patient.

In one embodiment, the coordinator 111 can determine the stimulation quantity proportion of the second left sacral nerve electrical pulse stimulation signal and the second right sacral nerve electrical pulse stimulation signal according to the second motor threshold and the second sensory threshold in real time. The second motor threshold and the second sensory threshold in real time can be the motor threshold and the sensory threshold manually input by the doctor or the motor threshold and the sensory threshold automatically obtained by the pulse generator 100. In one embodiment, the coordinator 111 can determine the stimulation quantity proportion of the second left sacral nerve electrical pulse stimulation signal and the second right sacral nerve electrical pulse stimulation signal according to the command received from the external programmer 300. The stimulation quantity proportion of the second left sacral nerve electrical pulse stimulation signal and the second right sacral nerve electrical pulse stimulation signal is different from the stimulation quantity proportion of the first left sacral nerve electrical pulse stimulation signal and the first right sacral nerve electrical pulse stimulation signal. The first left sacral nerve electrical pulse stimulation signal and the first right sacral nerve electrical pulse stimulation signal can be applied simultaneously. The first left sacral nerve electrical pulse stimulation signal and the first right sacral nerve electrical pulse stimulation signal can be applied alternately. The first left sacral nerve electrical pulse stimulation signal and the first right sacral nerve electrical pulse stimulation signal can be applied randomly. The first left sacral nerve electrical pulse stimulation signal and the first right sacral nerve electrical pulse stimulation signal can be applied orderly in a short term but disorderly in a long term.

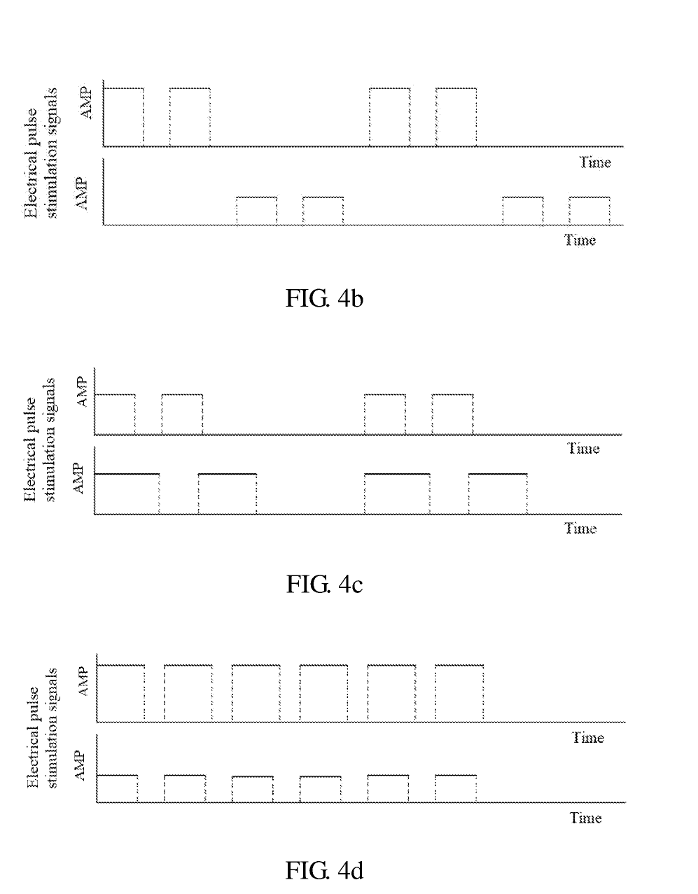

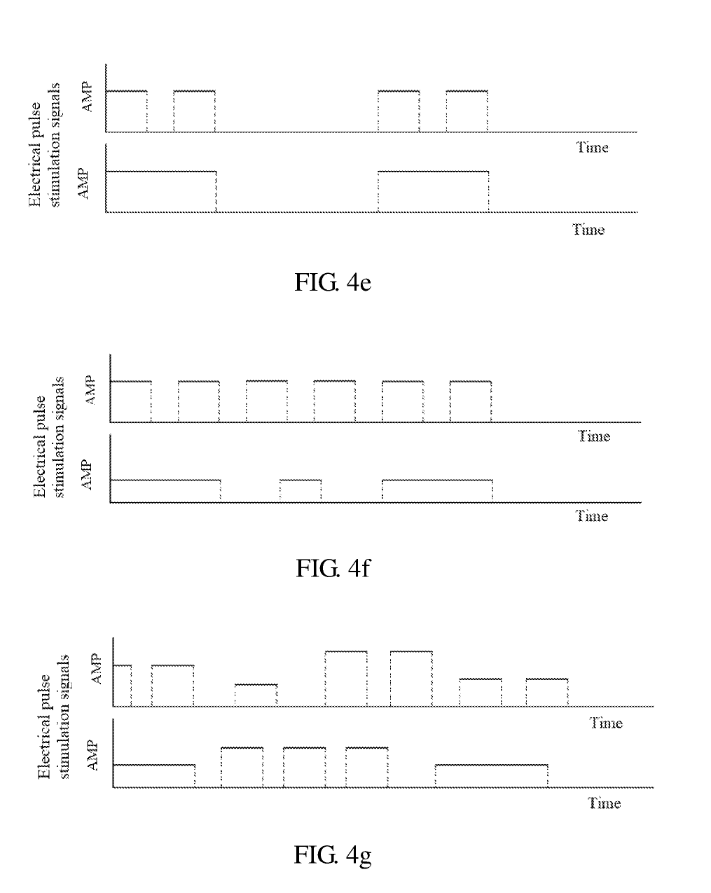

FIGS. 4a-4g show different kinds of electrical pulse stimulation signals generated by the coordinator 111. The two electrical pulse stimulation signals of each of FIGS. 4a-4g can be respectively applied to the left sacral nerve and the right sacral nerve. The electrical pulse stimulation signals generated by the coordinator 111 are not limited to the electrical pulse stimulation signals generated of FIGS. 4a-4g. If the motor threshold and the sensory threshold in real time are changed, the electrical pulse stimulation signals generated by the coordinator 111 can be adjusted according to need. For example, when it is detected that the second motor threshold and the second sensory threshold are changed to the third motor threshold and the third sensory threshold, the third left sacral nerve electrical pulse stimulation signal and the third right sacral nerve electrical pulse stimulation signal can be applied.

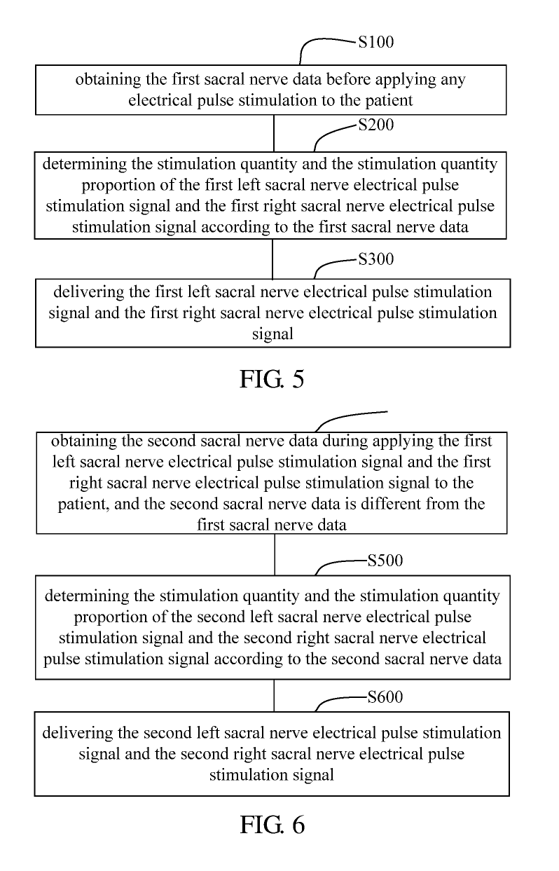

As shown in FIG. 5, a method for controlling the electrical pulse stimulation signal of the pulse generator 100 is provided. The method includes:

S100, obtaining the first sacral nerve data before applying any electrical pulse stimulation to the patient;

S200, determining the stimulation quantity and the stimulation quantity proportion of the first left sacral nerve electrical pulse stimulation signal and the first right sacral nerve electrical pulse stimulation signal according to the first sacral nerve data; and

S300, delivering the first left sacral nerve electrical pulse stimulation signal and the first right sacral nerve electrical pulse stimulation signal.

In S100, the first sacral nerve data includes the initial left sacral nerve sensory threshold, the initial right sacral nerve sensory threshold, the initial left sacral nerve motor threshold, and the initial right sacral nerve motor threshold. The first sacral nerve data is obtained by the pulse generator 100 via the communication device 140.

In S200, the stimulation quantity and the stimulation quantity proportion of the first left sacral nerve electrical pulse stimulation signal and the first right sacral nerve electrical pulse stimulation signal is determined by the coordinator 111. The first left sacral nerve electrical pulse stimulation signal and the first right sacral nerve electrical pulse stimulation signal are interrelated with each other. The relevance between the first left sacral nerve electrical pulse stimulation signal and the first right sacral nerve electrical pulse stimulation signal depends on the initial left sacral nerve sensory threshold, the initial right sacral nerve sensory threshold, the initial left sacral nerve motor threshold, and the initial right sacral nerve motor threshold.

In S300, in one embodiment, the first left sacral nerve electrical pulse stimulation signal is applied to the left sacral nerve via the first lead 201, and the first right sacral nerve electrical pulse stimulation signal is applied to the right sacral nerve via the second lead 202. In one embodiment, the first left sacral nerve electrical pulse stimulation signal and the first right sacral nerve electrical pulse stimulation signal are applied to the different nervous ramifications of the left sacral nerve right sacral nerve.

The method for controlling the electrical pulse stimulation signal of the pulse generator 100 is more accurate because the stimulation quantity proportion of the first left sacral nerve electrical pulse stimulation signal and the first right sacral nerve electrical pulse stimulation signal can better match the physiological function proportion of the patient and better simulate physiological dominance difference. The method for controlling the electrical pulse stimulation signal of the pulse generator 100 can synthetically adjust the electrical pulse stimulation signal according to the patients' nerve anatomy or function distribution variation.

The method of FIG. 5 can further include steps S400, S500 and S600 as shown in FIG. 6 after S300:

S400, obtaining the second sacral nerve data during applying the first left sacral nerve electrical pulse stimulation signal and the first right sacral nerve electrical pulse stimulation signal to the patient, and the second sacral nerve data is different from the first sacral nerve data;

S500, determining the stimulation quantity and the stimulation quantity proportion of the second left sacral nerve electrical pulse stimulation signal and the second right sacral nerve electrical pulse stimulation signal according to the second sacral nerve data; and

S600, delivering the second left sacral nerve electrical pulse stimulation signal and the second right sacral nerve electrical pulse stimulation signal.

In S400, the second sacral nerve data includes the physiological characteristic of the patient after the sacral nerve sensory threshold and the sacral nerve motor threshold are changed. The second sacral nerve data is different from the first sacral nerve data after the patient being stimulated for a period of time. The second sacral nerve data includes the real time left sacral nerve sensory threshold, the real time right sacral nerve sensory threshold, the real time left sacral nerve motor threshold, and the real time right sacral nerve motor threshold.

In S500, the stimulation quantity and the stimulation quantity proportion of the second left sacral nerve electrical pulse stimulation signal and the second right sacral nerve electrical pulse stimulation signal is determined by the coordinator 111. The second left sacral nerve electrical pulse stimulation signal and the second right sacral nerve electrical pulse stimulation signal are interrelated with each other. The relevance between the second left sacral nerve electrical pulse stimulation signal and the second right sacral nerve electrical pulse stimulation signal depends on the real time left sacral nerve sensory threshold, the real time right sacral nerve sensory threshold, the real time left sacral nerve motor threshold, and the real time right sacral nerve motor threshold.

In S600, in one embodiment, the second left sacral nerve electrical pulse stimulation signal is applied to the left sacral nerve via the first lead 201, and the second right sacral nerve electrical pulse stimulation signal is applied to the right sacral nerve via the second lead 202. In one embodiment, the second left sacral nerve electrical pulse stimulation signal and the second right sacral nerve electrical pulse stimulation signal are applied to the different nervous ramifications of the left sacral nerve or the right sacral nerve.

The method of FIG. 6 can adjust the left sacral nerve electrical pulse stimulation signal and the right sacral nerve electrical pulse stimulation signal according to the real time sacral nerve data. Thus, the therapy can be adjusted according to the patients' nerve anatomy or function distribution variation, and the therapy effect cam be improved.

In one embodiment, the stimulation quantity proportion of the second left sacral nerve electrical pulse stimulation signal and the second right sacral nerve electrical pulse stimulation signal can be the same as or different from the stimulation quantity proportion of the first left sacral nerve electrical pulse stimulation signal and the first right sacral nerve electrical pulse stimulation signal.

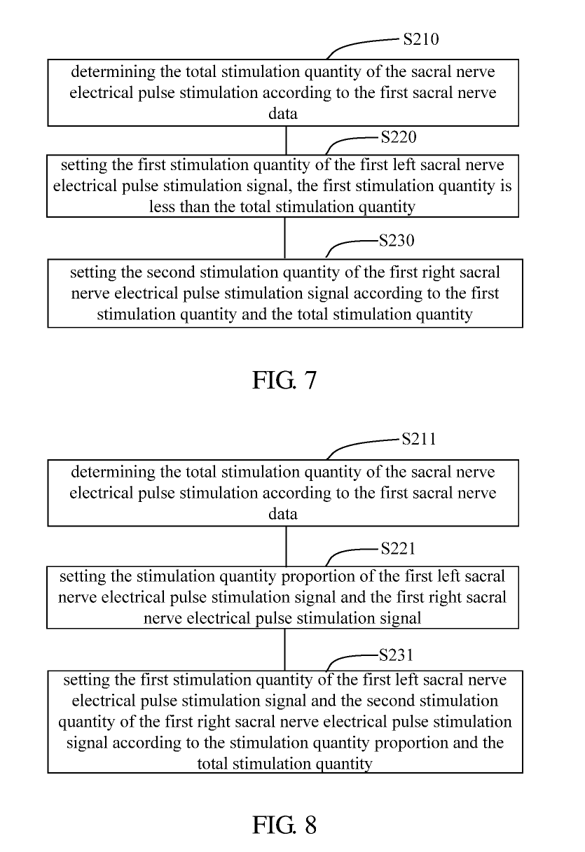

As shown in FIG. 7, in one embodiment, S200 can include:

S210, determining the total stimulation quantity of the sacral nerve electrical pulse stimulation according to the first sacral nerve data;

S220, setting the first stimulation quantity of the first left sacral nerve electrical pulse stimulation signal, the first stimulation quantity is less than the total stimulation quantity; and

S230, setting the second stimulation quantity of the first right sacral nerve electrical pulse stimulation signal according to the first stimulation quantity and the total stimulation quantity.

As shown in FIG. 8, in one embodiment, S200 can include:

S211, determining the total stimulation quantity of the sacral nerve electrical pulse stimulation according to the first sacral nerve data;

S221, setting the stimulation quantity proportion of the first left sacral nerve electrical pulse stimulation signal and the first right sacral nerve electrical pulse stimulation signal; and

S231, setting the first stimulation quantity of the first left sacral nerve electrical pulse stimulation signal and the second stimulation quantity of the first right sacral nerve electrical pulse stimulation signal according to the stimulation quantity proportion and the total stimulation quantity.

According to the method of FIG. 8, the total stimulation quantity of the sacral nerve electrical pulse stimulation applied to both the left sacral nerve and the right sacral nerve is set first, and then the stimulation quantity proportion of the sacral nerve electrical pulse stimulation applied to both the left sacral nerve and the right sacral nerve is determined. Thus, the total stimulation quantity of the sacral nerve electrical pulse stimulation applied to both the left sacral nerve and the right sacral nerve is controlled on the whole. On the condition of the stimulation quantity proportion, the total stimulation quantity can be kept in stable output by determining the total stimulation quantity first.

The methods of determining the stimulation quantity proportion is not limited to the methods of FIGS. 7-8 and can be performed by other methods.

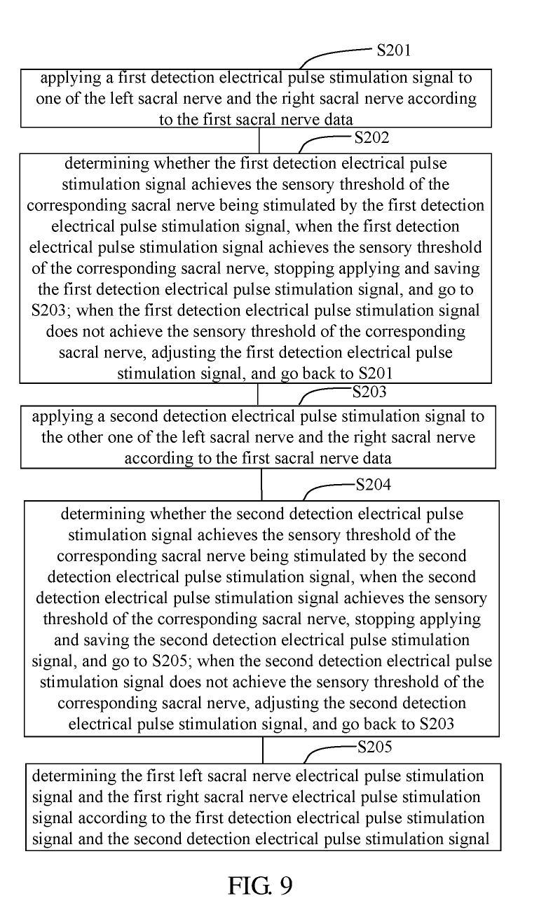

As shown in FIG. 9, in one embodiment, S200 can include:

S201, applying a first detection electrical pulse stimulation signal to one of the left sacral nerve and the right sacral nerve according to the first sacral nerve data;

S202, determining whether the first detection electrical pulse stimulation signal achieves the sensory threshold of the corresponding sacral nerve being stimulated by the first detection electrical pulse stimulation signal, when the first detection electrical pulse stimulation signal achieves the sensory threshold of the corresponding sacral nerve, stopping applying and saving the first detection electrical pulse stimulation signal, and go to S203; when the first detection electrical pulse stimulation signal does not achieve the sensory threshold of the corresponding sacral nerve, adjusting the first detection electrical pulse stimulation signal, and go back to S201;

S203, applying a second detection electrical pulse stimulation signal to the other one of the left sacral nerve and the right sacral nerve according to the first sacral nerve data;

S204, determining whether the second detection electrical pulse stimulation signal achieves the sensory threshold of the corresponding sacral nerve being stimulated by the second detection electrical pulse stimulation signal, when the second detection electrical pulse stimulation signal achieves the sensory threshold of the corresponding sacral nerve, stopping applying and saving the second detection electrical pulse stimulation signal, and go to S205; when the second detection electrical pulse stimulation signal does not achieve the sensory threshold of the corresponding sacral nerve, adjusting the second detection electrical pulse stimulation signal, and go back to S203; and

S205, determining the first left sacral nerve electrical pulse stimulation signal and the first right sacral nerve electrical pulse stimulation signal according to the first detection electrical pulse stimulation signal and the second detection electrical pulse stimulation signal.

In S201, the first detection electrical pulse stimulation signal is selected according to the sensory threshold of the first sacral nerve data, and the total stimulation quantity and/or the stimulation quantity proportion. The current and voltage of the first detection electrical pulse stimulation signal should be slightly higher than the weakest electrical pulse stimulation that the patient can feel. The motor threshold is usually higher than the sensory threshold. When the first detection electrical pulse stimulation signal achieves the motor threshold, the patient would feel uncomfortable because of the too strong electrical pulse stimulation.

In S202, the first detection electrical pulse stimulation signal achieves the sensory threshold refers that the current and voltage of the first detection electrical pulse stimulation signal is slightly higher than the weakest electrical pulse stimulation that the patient can feel. When the first detection electrical pulse stimulation signal does not achieve the sensory threshold of the corresponding sacral nerve, the stimulation quantity of the first detection electrical pulse stimulation signal are changed, and then go back to S201. According to S202, the stimulation quantity of the first detection electrical pulse stimulation signal can be changed many times until the first detection electrical pulse stimulation signal achieve the sensory threshold of the corresponding sacral nerve.

In S203, the second detection electrical pulse stimulation signal is selected according to the sensory threshold of the first sacral nerve data and the stimulation quantity of the first detection electrical pulse stimulation signal which achieves the sensory threshold of the corresponding sacral nerve.

In S204, the second detection electrical pulse stimulation signal achieves the sensory threshold refers that the current and voltage of the second detection electrical pulse stimulation signal is slightly higher than the weakest electrical pulse stimulation the corresponding sacral nerve of the patient can feel. When the second detection electrical pulse stimulation signal does not achieve the sensory threshold of the corresponding sacral nerve, the stimulation quantity of the second detection electrical pulse stimulation signal are changed, and then go back to S203. According to S204, the stimulation quantity of the second detection electrical pulse stimulation signal can be changed many times until the second detection electrical pulse stimulation signal achieve the sensory threshold of the corresponding sacral nerve.

In S205, the first detection electrical pulse stimulation signal, that achieves the sensory threshold of the left sacral nerve, is used as the he first left sacral nerve electrical pulse stimulation signal; and the second detection electrical pulse stimulation signal, that achieves the sensory threshold of the right sacral nerve, is used as the he first right sacral nerve electrical pulse stimulation signal.

In one embodiment, the first electrical pulse stimulation signal applied to the left sacral nerve and the electrical pulse stimulation signal applied to the right sacral nerve can be determined according to the left sacral nerve sensory threshold and the right sacral nerve sensory threshold. The left sacral nerve sensory threshold and the right sacral nerve sensory threshold can be the initial threshold before the patient being stimulated or real time threshold after the patient being stimulated.

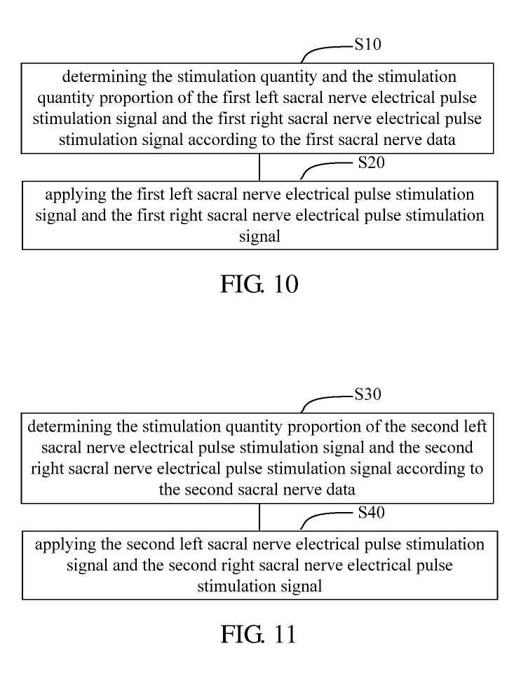

As shown in FIG. 10, a method for controlling the electrical pulse stimulation system 10 is provided. The electrical pulse stimulation system 10 includes the coordinator 111. The pulse applying method of the coordinator 111 includes:

S10, determining the stimulation quantity and the stimulation quantity proportion of the first left sacral nerve electrical pulse stimulation signal and the first right sacral nerve electrical pulse stimulation signal according to the first sacral nerve data; and

S20, applying the first left sacral nerve electrical pulse stimulation signal and the first right sacral nerve electrical pulse stimulation signal.

The first sacral nerve data can be physiological characteristic of the patients obtained by the pulse generator 100 from the external programmer 300. The stimulation quantity proportion of the first left sacral nerve electrical pulse stimulation signal and the first right sacral nerve electrical pulse stimulation signal can be automatically adjusted by machine learning of the pulse generator 100. The stimulation quantity proportion of the first left sacral nerve electrical pulse stimulation signal and the first right sacral nerve electrical pulse stimulation signal can be manually adjusted via the external programmer 300 by the doctor.

In one embodiment, the first left sacral nerve electrical pulse stimulation signal and the first right sacral nerve electrical pulse stimulation signal can be applied in a preset stimulation sequence. For example, the first left sacral nerve electrical pulse stimulation signal and the first right sacral nerve electrical pulse stimulation signal can be applied simultaneously. For example, the first left sacral nerve electrical pulse stimulation signal and the first right sacral nerve electrical pulse stimulation signal can be applied alternately. For example, the first left sacral nerve electrical pulse stimulation signal and the first right sacral nerve electrical pulse stimulation signal can be applied randomly. The first left sacral nerve electrical pulse stimulation signal and the first right sacral nerve electrical pulse stimulation signal can be applied orderly in a short term but disorderly in a long term.

In one embodiment, the first left sacral nerve electrical pulse stimulation signal and the first right sacral nerve electrical pulse stimulation signal can be applied to the same nervous ramifications of the left sacral nerve and the right sacral nerve. For example, the first left sacral nerve electrical pulse stimulation signal is applied to the left S2 sacral nerve via the first lead 201, and the first right sacral nerve electrical pulse stimulation signal is applied to the right S2 sacral nerve via the second lead 202.

In one embodiment, the first left sacral nerve electrical pulse stimulation signal and the first right sacral nerve electrical pulse stimulation signal can be applied to the different nervous ramifications of the left sacral nerve and the right sacral nerve. For example, the first left sacral nerve electrical pulse stimulation signal is applied to the left S2 sacral nerve via the first lead 201, and the first right sacral nerve electrical pulse stimulation signal is applied to the right S4 sacral nerve via the second lead 202.

As shown in FIG. 11, the method of FIG. 10 can further include steps S30 and S40:

S30, determining the stimulation quantity proportion of the second left sacral nerve electrical pulse stimulation signal and the second right sacral nerve electrical pulse stimulation signal according to the second sacral nerve data; and

S40, applying the second left sacral nerve electrical pulse stimulation signal and the second right sacral nerve electrical pulse stimulation signal.

In one embodiment, the second left sacral nerve electrical pulse stimulation signal and the second right sacral nerve electrical pulse stimulation signal can be applied in a preset stimulation sequence.

In one embodiment, the second left sacral nerve electrical pulse stimulation signal and the second right sacral nerve electrical pulse stimulation signal can be applied to the same nervous ramifications of the left sacral nerve and the right sacral nerve. For example, the second left sacral nerve electrical pulse stimulation signal is applied to the left S3 sacral nerve by implanting the first lead 201 in the left S3 sacral nerve hole, and the second right sacral nerve electrical pulse stimulation signal is applied to the right S3 sacral nerve by implanting the second lead 202 in the right S3 sacral nerve hole.

In one embodiment, the second left sacral nerve electrical pulse stimulation signal and the second right sacral nerve electrical pulse stimulation signal can be applied to the different nervous ramifications of the left sacral nerve and the right sacral nerve. For example, the second left sacral nerve electrical pulse stimulation signal is applied to the left S2 sacral nerve by implanting the first lead 201 in the left S2 sacral nerve hole, and the second right sacral nerve electrical pulse stimulation signal is applied to the right S3 sacral nerve by implanting the second lead 202 in the right S3 sacral nerve hole. In another example, the second left sacral nerve electrical pulse stimulation signal is applied to the left S3 sacral nerve by implanting the first lead 201 in the left S3 sacral nerve hole, and the second right sacral nerve electrical pulse stimulation signal is applied to the right S4 sacral nerve by implanting the second lead 202 in the right S4 sacral nerve hole.

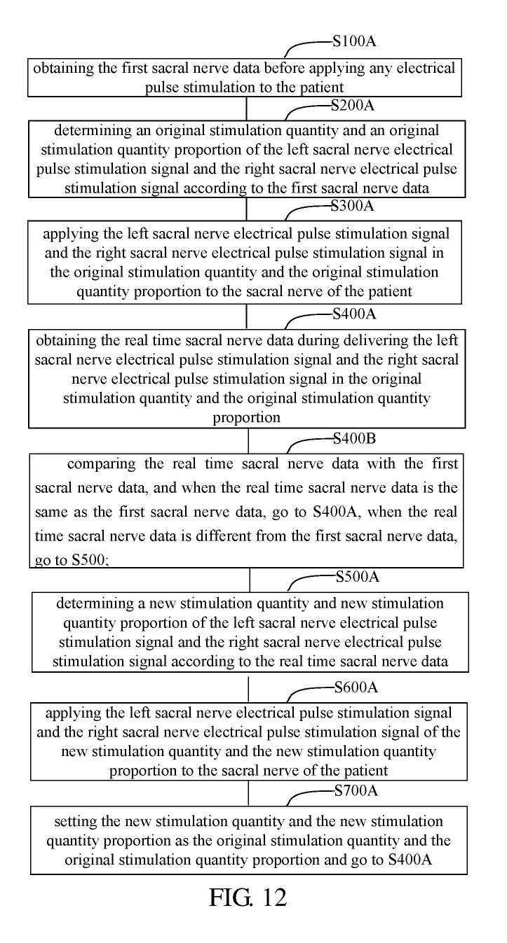

As shown in FIG. 12, an electrical pulse stimulation therapy for urinary dysfunction is provided. The method is performed by the electrical pulse stimulation system 10 and includes:

S100A, obtaining the first sacral nerve data before applying any electrical pulse stimulation to the patient;

S200A, determining an original stimulation quantity and an original stimulation quantity proportion of the left sacral nerve electrical pulse stimulation signal and the right sacral nerve electrical pulse stimulation signal according to the first sacral nerve data;

S300A, applying the left sacral nerve electrical pulse stimulation signal and the right sacral nerve electrical pulse stimulation signal in the original stimulation quantity and the original stimulation quantity proportion to the sacral nerve of the patient;

S400A, obtaining the real time sacral nerve data during delivering the left sacral nerve electrical pulse stimulation signal and the right sacral nerve electrical pulse stimulation signal in the original stimulation quantity and the original stimulation quantity proportion;

S400B, comparing the real time sacral nerve data with the first sacral nerve data, and when the real time sacral nerve data is the same as the first sacral nerve data, go to S400A, when the real time sacral nerve data is different from the first sacral nerve data, go to S500;

S500A, determining a new stimulation quantity and new stimulation quantity proportion of the left sacral nerve electrical pulse stimulation signal and the right sacral nerve electrical pulse stimulation signal according to the real time sacral nerve data;

S600A, applying the left sacral nerve electrical pulse stimulation signal and the right sacral nerve electrical pulse stimulation signal of the new stimulation quantity and the new stimulation quantity proportion to the sacral nerve of the patient; and

S700A, setting the new stimulation quantity and the new stimulation quantity proportion as the original stimulation quantity and the original stimulation quantity proportion and go to S400A.

It is to be understood that the above-described exemplary embodiments are intended to illustrate rather than limit the disclosure. Any elements described in accordance with any exemplary embodiments is understood that they can be used in addition or substituted in other exemplary embodiments. Exemplary embodiments can also be used together. Variations may be made to the exemplary embodiments without departing from the spirit of the disclosure. The above-described exemplary embodiments illustrate the scope of the disclosure but do not restrict the scope of the disclosure.

Depending on the exemplary embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

What is claimed is:

1. An electrical pulse stimulation system, comprising:
a pulse generator;
a first lead electrically connected to the pulse generator; and
a second lead electrically connected to the pulse generator;
wherein the pulse generator is configured to deliver a left sacral nerve electrical pulse stimulation signal to the first lead and deliver a right sacral nerve electrical pulse stimulation signal to the second lead; and the pulse generator is further configured to determine and adjust a stimulation quantity proportion of the left sacral nerve electrical pulse stimulation signal and the right sacral nerve electrical pulse stimulation signal according to a sacral nerve data of a patient.

2. The electrical pulse stimulation system of claim 2, further comprising an external programmer, wherein the external programmer communicates with the pulse generator and is configured to send an initial sacral nerve data to the pulse generator.

3. The electrical pulse stimulation system of claim 2, wherein the pulse generator comprises:
a processor;
a pulse generation circuit electrically connected to the processor;
a communication device electrically connected to the processor;
a memory electrically connected to the processor; and
a coordinator electrically connected to the processor, the first lead, and the second lead;
wherein the coordinator is configured to determine a first stimulation quantity proportion of a first left sacral nerve electrical pulse stimulation signal and a first right sacral nerve electrical pulse stimulation signal according to the initial sacral nerve data of the patient, and deliver the first left sacral nerve electrical pulse stimulation signal to the first lead and deliver the first right sacral nerve electrical pulse stimulation signal to the second lead.

4. The electrical pulse stimulation system of claim 3, wherein the pulse generator further comprises a sensor, the sensor is electrically connected to the processor and configured to detect a real time sacral nerve data of the patient;
wherein the coordinator is further configured to determine a second stimulation quantity proportion of a second left sacral nerve electrical pulse stimulation signal and a second right sacral nerve electrical pulse stimulation signal according to the real time sacral nerve data of the patient, and deliver the second left sacral nerve electrical pulse stimulation signal to the first lead and deliver the second right sacral nerve electrical pulse stimulation signal to the second lead.

5. The electrical pulse stimulation system of claim 4, wherein the coordinator has a machine learning function so that the coordinator is configured to automatically adjust the second stimulation quantity proportion of the second left sacral nerve electrical pulse stimulation signal and the second right sacral nerve electrical pulse stimulation signal according to the real time sacral nerve data of the patient.

6. The electrical pulse stimulation system of claim 5, wherein the second stimulation quantity proportion is different from the first stimulation quantity proportion.

7. The electrical pulse stimulation system of claim 3, wherein the coordinator is further configured to apply the first and second left sacral nerve electrical pulse stimulation signals and the first and second right sacral nerve electrical pulse stimulation signals in a preset stimulation sequence.

8. The electrical pulse stimulation system of claim 7, wherein the coordinator is further configured to simultaneously apply the first and second left sacral nerve electrical pulse stimulation signals and the first and second right sacral nerve electrical pulse stimulation signals.

9. The electrical pulse stimulation system of claim 7, wherein the coordinator is further configured to alternately apply the first and second left sacral nerve electrical pulse stimulation signals and the first and second right sacral nerve electrical pulse stimulation signals.

10. The electrical pulse stimulation system of claim 7, wherein the coordinator is further configured to randomly apply the first and second left sacral nerve electrical pulse stimulation signals and the first and second right sacral nerve electrical pulse stimulation signals.

11. A method for controlling a pulse generator of an electrical pulse stimulation system, comprising:
obtaining an initial sacral nerve data;
determining a first left sacral nerve electrical pulse stimulation signal and a first right sacral nerve electrical pulse stimulation signal according to the initial sacral nerve data; and
applying the first left sacral nerve electrical pulse stimulation signal and the first right sacral nerve electrical pulse stimulation signal.

12. The method of claim 11, wherein further comprising:
obtaining a real time sacral nerve data during applying the first left sacral nerve electrical pulse stimulation signal and the first right sacral nerve electrical pulse stimulation signal, and the real time sacral nerve data is different from the initial sacral nerve data;
determining a second left sacral nerve electrical pulse stimulation signal and a second right sacral nerve electrical pulse stimulation signal according to the real time sacral nerve data; and
applying the second left sacral nerve electrical pulse stimulation signal and the second right sacral nerve electrical pulse stimulation signal.

13. The method of claim 11, wherein the determining the first left sacral nerve electrical pulse stimulation signal and the first right sacral nerve electrical pulse stimulation signal according to the initial sacral nerve data comprises:
determining the total stimulation quantity of the sacral nerve electrical pulse stimulation according to the initial sacral nerve data;
setting a first stimulation quantity of the first left sacral nerve electrical pulse stimulation signal, wherein the first stimulation quantity is less than the total stimulation quantity; and
setting a second stimulation quantity of the first right sacral nerve electrical pulse stimulation signal according to the first stimulation quantity and the total stimulation quantity.

14. The method of claim 11, wherein the determining the first left sacral nerve electrical pulse stimulation signal and the first right sacral nerve electrical pulse stimulation signal according to the initial sacral nerve data comprises:
determining the total stimulation quantity of the sacral nerve electrical pulse stimulation according to the initial sacral nerve data;
setting a first stimulation quantity proportion of the first left sacral nerve electrical pulse stimulation signal and the first right sacral nerve electrical pulse stimulation signal; and
setting a first stimulation quantity of the first left sacral nerve electrical pulse stimulation signal and a second stimulation quantity of the first right sacral nerve electrical pulse stimulation signal according to the first stimulation quantity proportion and the total stimulation quantity.

15. The method of claim 11, wherein the determining the first left sacral nerve electrical pulse stimulation signal and the first right sacral nerve electrical pulse stimulation signal according to the initial sacral nerve data comprises:
S201, applying a first detection electrical pulse stimulation signal to one of a left sacral nerve and a right sacral nerve according to the initial sacral nerve data;
S202, determining whether the first detection electrical pulse stimulation signal achieves a sensory threshold of the corresponding sacral nerve being stimulated by the first detection electrical pulse stimulation signal, when the first detection electrical pulse stimulation signal achieves the sensory threshold of the corresponding sacral nerve, stopping applying and saving the first detection electrical pulse stimulation signal, and go to S203; when the first detection electrical pulse stimulation signal does not achieve the sensory threshold of the corresponding sacral nerve, adjusting the first detection electrical pulse stimulation signal, and go back to S201;
S203, applying a second detection electrical pulse stimulation signal to the other one of the left sacral nerve and the right sacral nerve according to the first sacral nerve data;
S204, determining whether the second detection electrical pulse stimulation signal achieves the sensory threshold of the corresponding sacral nerve being stimulated by the second detection electrical pulse stimulation signal, when the second detection electrical pulse stimulation signal achieves the sensory threshold of the corresponding sacral nerve, stopping applying and saving the second detection electrical pulse stimulation signal, and go to S205; when the second detection electrical pulse stimulation signal does not achieve the sensory threshold of the corresponding sacral nerve, adjusting the second detection electrical pulse stimulation signal, and go back to S203; and
S205, determining the first left sacral nerve electrical pulse stimulation signal and the first right sacral nerve electrical pulse stimulation signal according to the first detection electrical pulse stimulation signal and the second detection electrical pulse stimulation signal.

16. An electrical pulse stimulation therapy for urinary dysfunction by an electrical pulse stimulation system, comprising:
obtaining an initial sacral nerve data before applying any electrical pulse stimulation to the patient;
determining a first left sacral nerve electrical pulse stimulation signal and a first right sacral nerve electrical pulse stimulation signal according to the initial sacral nerve data; and
applying the first left sacral nerve electrical pulse stimulation signal and the first right sacral nerve electrical pulse stimulation signal to sacral nerve of the patient.

17. The therapy of claim 11, wherein further comprising:
obtaining a real time sacral nerve data during applying the first left sacral nerve electrical pulse stimulation signal and the first right sacral nerve electrical pulse stimulation signal to the sacral nerve of the patient, and the real time sacral nerve data is different from the initial sacral nerve data;
determining a second left sacral nerve electrical pulse stimulation signal and a second right sacral nerve electrical pulse stimulation signal according to the real time sacral nerve data; and
applying the second left sacral nerve electrical pulse stimulation signal and the second right sacral nerve electrical pulse stimulation signal to the sacral nerve of the patient.

18. The therapy of claim 11, wherein the determining the first left sacral nerve electrical pulse stimulation signal and the first right sacral nerve electrical pulse stimulation signal according to the initial sacral nerve data comprises:
determining the total stimulation quantity of the sacral nerve electrical pulse stimulation according to the initial sacral nerve data;
setting a first stimulation quantity of the first left sacral nerve electrical pulse stimulation signal, wherein the first stimulation quantity is less than the total stimulation quantity; and
setting a second stimulation quantity of the first right sacral nerve electrical pulse stimulation signal according to the first stimulation quantity and the total stimulation quantity.

19. The therapy of claim 11, wherein the determining the first left sacral nerve electrical pulse stimulation signal and the first right sacral nerve electrical pulse stimulation signal according to the initial sacral nerve data comprises:
determining the total stimulation quantity of the sacral nerve electrical pulse stimulation according to the initial sacral nerve data;
setting a first stimulation quantity proportion of the first left sacral nerve electrical pulse stimulation signal and the first right sacral nerve electrical pulse stimulation signal; and
setting a first stimulation quantity of the first left sacral nerve electrical pulse stimulation signal and a second stimulation quantity of the first right sacral nerve electrical pulse stimulation signal according to the first stimulation quantity proportion and the total stimulation quantity.

20. The therapy of claim 11, wherein the determining the first left sacral nerve electrical pulse stimulation signal and the first right sacral nerve electrical pulse stimulation signal according to the initial sacral nerve data comprises:
S201, applying a first detection electrical pulse stimulation signal to one of a left sacral nerve and a right sacral nerve according to the initial sacral nerve data;
S202, determining whether the first detection electrical pulse stimulation signal achieves a sensory threshold of the corresponding sacral nerve being stimulated by the first detection electrical pulse stimulation signal, when the first detection electrical pulse stimulation signal achieves the sensory threshold of the corresponding sacral nerve, stopping applying and saving the first detection electrical pulse stimulation signal, and go to S203; when the first detection electrical pulse stimulation signal does not achieve the sensory threshold of the corresponding sacral nerve, adjusting the first detection electrical pulse stimulation signal, and go back to S201;
S203, applying a second detection electrical pulse stimulation signal to the other one of the left sacral nerve and the right sacral nerve according to the first sacral nerve data;
S204, determining whether the second detection electrical pulse stimulation signal achieves the sensory threshold of the corresponding sacral nerve being stimulated by the second detection electrical pulse stimulation signal, when the second detection electrical pulse stimulation signal achieves the sensory threshold of the corresponding sacral nerve, stopping applying and saving the second detection electrical pulse stimulation signal, and go to S205; when the second detection electrical pulse stimulation signal does not achieve the sensory threshold of the corresponding sacral nerve, adjusting the second detection electrical pulse stimulation signal, and go back to S203; and
S205, determining the first left sacral nerve electrical pulse stimulation signal and the first right sacral nerve electrical pulse stimulation signal according to the first detection electrical pulse stimulation signal and the second detection electrical pulse stimulation signal.
